(19) United States
(12) Patent Application Publication
(10) Pub. No.: US 2003/0236449 A1
(43) Pub. Date: Dec. 25, 2003

(54) Paediatric electronic device

(76) Inventor: Maurice Kelvin Naidoo, Johannesburg (ZA)

Correspondence Address: MAURICE KELVIN NAIDOO, POSTNET SUITE 118, X2600, HOUGHTON, JOHANNESBURG, 2041, ZA

(21) Appl. No.: 10/176,415

(22) Filed: Jun. 20, 2002

(51) Int. Cl. A61B 5/00

(52) U.S. Cl. 600/300

(57) ABSTRACT

A paediatric electronic device includes a housing, a length measuring means for measuring the length of a young child, a memory means for storing data in regard to various weight dependent parameters, a processing means for determining the value of at least one of the parameters in accordance with the length of the young child as measured by the length measuring means, and a display means for displaying the values of the parameters.

This invention relates to a paediatric electronic device. More particularly, this invention relates to a paediatric electronic device whereby a person is provided with information relating to a young child. The person may be a doctor or parent and the information may relate to physical measurements, suitable drugs, dosages, medical procedures, equipment, milestones and clothing.

In this specification the term “young child” includes a baby.

According to the invention there is provided a paediatric electronic device which includes

a housing;

a length measuring means for measuring the length of a young child;

a memory means for storing data in regard to various weight dependent parameters;

a processing means for determining the value of at least one of the parameters in accordance with the length of the young child as measured by the length measuring means; and

a display means for displaying the values of the parameters.

Conveniently, the parameters may be grouped in various groups and the device may include a group selecting means for selecting which group of parameters is displayed.

The parameters may relate to physical measurements, suitable drugs, dosages, medical procedures, equipment, milestones or clothing.

The particular values of the various parameters for each length range may be stored in a data base or may be computed in accordance with a formula, stored in the memory means.

The memory means may store a length of a young child that has been measured, until reset.

The measuring means may include a length of tape housed in the housing and extractable therefrom, with an electronically operable monitoring means for monitoring the extent to which the tape has been extracted from the housing. A retraction means may also be provided to automatically retract the tape into the housing. Preferably a locking means is provided to lock the measuring tape when it has been extracted to a desired extent.

The memory means may be reset when the measuring tape is extracted from the housing after having been retracted into the housing.

The monitoring means may include an optical sensor responsive to formations on the tape, the number of formations passing past the sensor being counted. The formations may be openings at spaced intervals in the tape.

The group selecting means may include a plurality of buttons which are selectively depressed by the person using the device to select which group of parameters is to be displayed.

The device may include a power supply and a back-up power supply. The device may further have a “sleep” function whereby power consumption is reduced if the device is not operated for a specified time period. Further, a low voltage monitor may be included together with a low voltage warning annunciator.

The display means may include a back-lit LCD display matrix with suitable drivers.

The invention is now described, by way of an example, with reference to the accompanying drawings, in which:

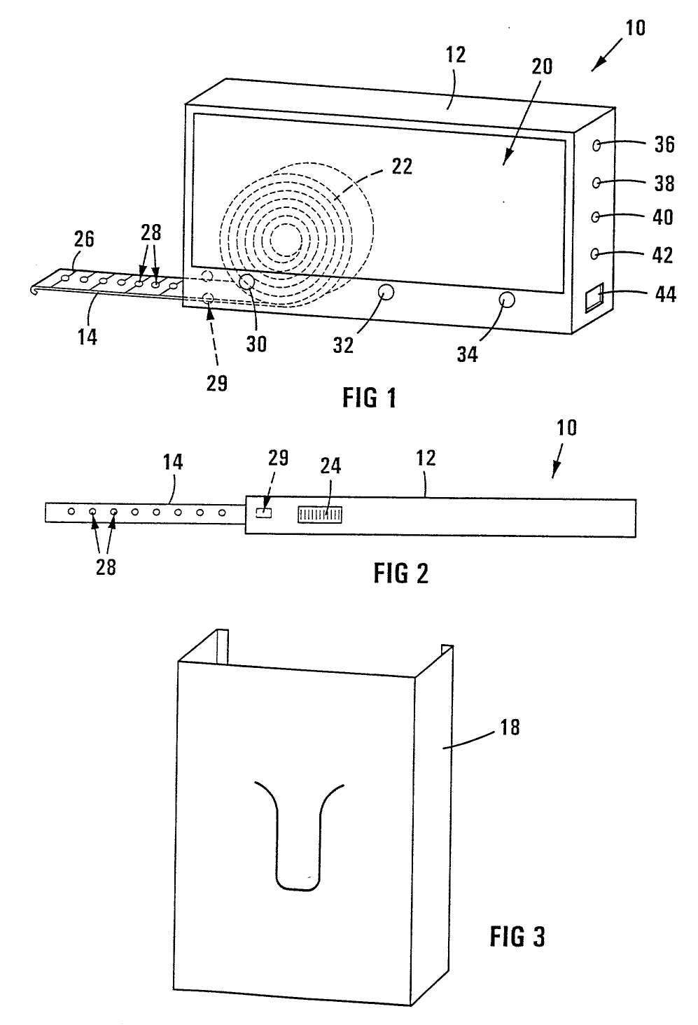

FIG. 1 shows a schematic front perspective view of a paediatric electronic device in accordance with the invention;

FIG. 2 shows a bottom view of the device;

FIG. 3 shows a perspective view of a holder for the device;

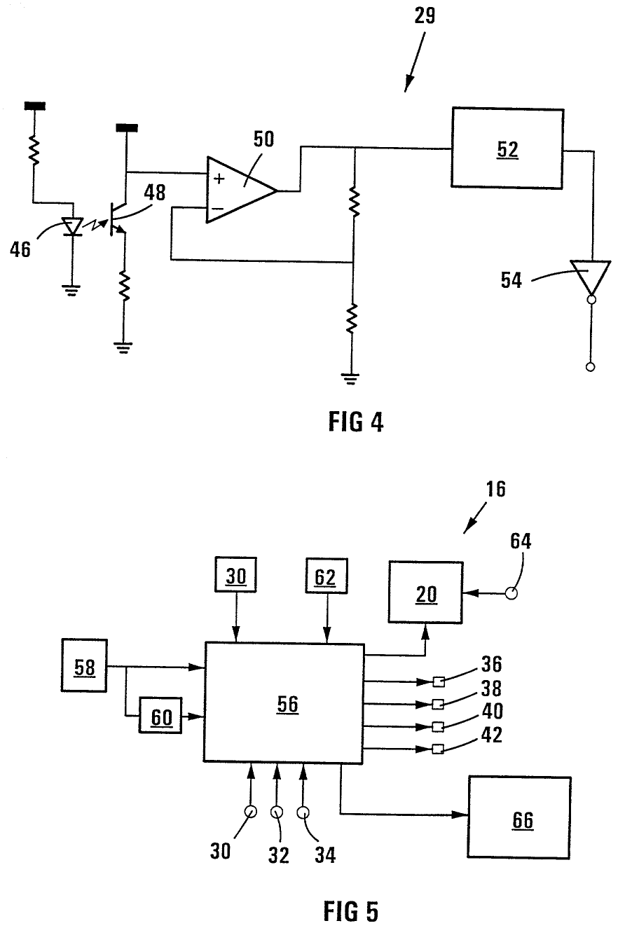

FIG. 4 shows a circuit diagram of a monitoring circuit for monitoring the extent to which a tape of the device is extracted; and

FIG. 5 shows a schematic block diagram of electronic componentry of the device.

Referring now to FIG. 1 of the drawings, a paediatric electronic device in accordance with the invention is designated generally by reference numeral 10. The device 10 has a housing 12 in which are housed a flexible metal measuring tape 14 and electronic componentry 16. The electronic componentry will be described hereinbelow with reference to FIG. 5. The housing 12 is rectangular and is of a similar size to pagers, such that it can be held in the hand of a user and control buttons operated, and can be carried in a holder, such as the holder 18 shown in FIG. 3, on the belt of a user or in a coat pocket.

The housing 12 has mounted therein a back-lit LCD display 20 on which the values of various parameters are displayed, depending on the extent to which the tape 14 is extracted, as will also be described more fully below.

The tape 14 is coiled within the housing 12 on a spool that is spring loaded by means of a coil spring 22 so that the tape 14 may be extracted a desired extent and is automatically retracted. A lock, which is operated frictionally by a slide 24, locks the tape 14 in a desired extracted extent.

The tape 14 has gradations 26 thereon and a number of central spaced apertures 28. The electronic componentry has an optical sensor 29 which senses the passage of the apertures 28, thereby to monitor the extent to which the tape 14 has been extracted.

On the front of the housing 12, below the display 20, there are three control buttons 30, 32 and 34. These control the parameters displayed, as will be described below.

Four differently coloured LED's 36, 38, 40 and 42 are mounted on the right side of the housing 12. The tape 14 is divided into seven sectors and the appropriate LED is energised, depending on the extent to which the tape 14 has been extended, to indicate a range. An “on/off” switch button 44 is also located on the right side of the housing 12.

A battery (not shown) is also housed in the housing 12 to provide power for the electronic componentry. A back-up battery (also not shown) is also provided.

Referring now to FIG. 4, the optical sensor 29 is shown. The sensor 29 comprises an LED 46 and phototransistor 48, which drives an op-amp 50. The op-amp 50 is connected to an inverted Schmitt trigger 52 which drives an inverter 54. The LED 46 is positioned on one side of the tape 14 and the phototransistor 48 on the other side, so that when an aperture 28 is aligned therewith, the light from the LED 46 is detected by the phototransistor and the Schmitt trigger 52 operated. The inverter 54 thus provides a series of signals to a processor (described below) as the tape 14 is moved past the sensor 29.

The electronic componentry is now described with reference to FIG. 5. The componentry includes a processor 56 which is supplied with power from the battery 58. A low voltage monitor 60 monitors the condition of the battery 58 and the processor 56 responds to a low voltage condition by displaying a suitable message on the display 20, or an LED (not shown). The sensor 29 is connected to the processor 56 and provides it with tape extension signals as described above. A reset signal is supplied,from a reset switch 62. The processor 56 is also connected to the range indicating LED's 36, 38, 40 and 42, as are the control buttons 30, 32 and 34. A further press-button switch 64 operates back-lighting of the display 20. Finally, the componentry also includes a memory module 66. The memory module 66 stores any program required for the processor as well as the relevant data required by the processor to display the values of the desired parameters on the display 20 in accordance with the extent to which the tape has been extracted.

The various parameters that are displayed, and illustrative values are now described by listing the various screen displays that are provided in the exemplary device. It is to be noted that the bottom line of each display is for control purposes and the expressions therein are associated with the appropriate control button 30, 32 or 34 aligned therewith.

FIRST SCREEN:

| | |
|---|---|
| AGE: | 8 YRS |
| WEIGHT: | 24 KG |

SELECT OPTION?
NVALU/EQUIP/DRUG

SECOND SCREEN:

| NVALU | PHYSICAL |
|---|---|
| RR | 15-26 b/m |
| PR | 60-140/60-90 |
| BP | 97-112/57-71 |
| T⁰ OR | 35.6-37.6 dc |
| AX | 35.2-37.2 dc |
| RE | 35.7-37.8 dc |

BIOC/XR ECG/MAIN

THIRD SCREEN:

| EQUIP | VENT SET |
|---|---|
| O2 | 100% |
| TV | 240-360 ml/kg |
| IT | 0.6 SEC |
| PIP | 20-30 cmH2O |
| VR | 15-20 b/m |
| PEEP | 3-5 cmH2O |
| IV ETC/AIR/MAIN | |

FOURTH SCREEN

| DRUG | INFUSIONS |
|---|---|
| EPI | 1.4 mg in 100 |
| NOR ml@ | 5-25 ml/hr |
| DOP | 144 mg in 100 |
| DOB ml@ | 5-20 ml/hr |
| LID | 288 mg in 100 |
| ml@ | 10-25 ml/hr |
| RESUS1/RES2/MAIN | |

FIFTH SCREEN:

| NVALU | BIOCHEM |
|---|---|
| PH | 7.35-7.45 |
| PaCO2 | 32-48 mmHg |
| PaO2 | 80-105 mmHg |
| Hb | 13.5 g/dl |
| UREA/Cr | 27-62 μmol/l |
| PHYS/XR ECG/MAIN | |

SIXTH SCREEN:

| NVALU | XR ECG |
|---|---|
| PR | >0.1 sec |
| QRS DURATION | 0.07 s |
| QRS/P AXIS | 60/0-90 |
| CSP ODO SPA | ≦3 mm |
| PRE VER | ≦5 mm |
| COR DIA | ≧13 mm |
| BIOCHM/PHYS/MAIN | |

SEVENTH SCREEN:

| EQUIP | HIV ETC |
|---|---|
| JELCO | 18-20 G |
| B/FLY | 21-22 G |
| N G T | 14-18 F |
| U CAT | 12 F |
| I C D | 28-32 F |
| VENT/AIRWAY/MAIN | |

EIGHTH SCREEN:

| EQUIP | AIR WAY |
|---|---|
| ETT UNCU | 6.0 mm CUFFED |
| T-T cm | 17-18 cm |
| L/SCOPE | 2-3 STR |
| | OR CURVE |
| STYLET | 14 F |
| SUCTION CAT | 10 F |
| VENT/IV ETC/MAIN | |

NINTH SCREEN:

| DRUG | RESUS1 | |
|---|---|---|
| EPI | 1:10000 | 2.4 ml |
| EPI | 1:1000 | 4.8 ml |
| ATR | 0.01% | 4.8 ml |
| BIC | 24 ml mEQ | 24 ml |
| LID | 2% | 1-2 ml |
| DFIB | | 48 J/96 J |

INFU/RESUS2/MAIN

TENTH SCREEN:

| DRUG | RESUS2 | |
|---|---|---|
| CALCI | 1% | 4.8 ml |
| FUROS | | 20 mg |
| DIAZE | | 2.4-7.2 mg |
| PHENO | | 360-480 mg |
| D2SW | 25% | 48 ml |
| LORAZ | | 2.4 mg |

INFU/RESUS1/MAIN

The abbreviated expressions in the screen displays have the following meanings:

| | |
|---|---|
| AGE - | Age |
| AIR - | Airway |
| AIRWAY - | Airway |
| ATR - | Atropine |
| B/FLY - | Butterfly |
| BIC - | Bicarbonate |
| BIOC - | Biochemistry |
| BIOCHM - | Biochemistry |
| BP - | Blood pressure |
| COR DIA - | Cord diameter |
| CDO SPA - | Odontoid space |
| CXR - | Chest x-ray |
| DFIB - | Defibrillation |
| DIAZE - | Diazepam |
| DOP - | Dopamine |
| DRUG - | Drug (or medication) |
| DRUG INFUSIONS - | Drug infusions |
| D2SW - | 25% dextrose water |
| EPI - | Epinephrine |
| EQUIP AIR WAY - | Equipment airway |
| EQUIP IV ETC - | Equipment intravenous, et cetera |
| EQUIP VENT SET - | Equipment ventilatory settings |
| ETT - | Endotracheal tube |
| FUROS - | Furosemide |
| CALCI - | Calcium |
| Hb - | Haemoglobin |
| I C D - | Intercostal drain |
| INFU - | Infusions |
| IT - | Inspiratory time |
| IV ETC - | Intravenous et cetera |
| JELCO - | Jelco |
| LID - | Lidocaine |
| LORAZ - | Lorazepam |
| MAIN - | Main menu |
| mEQ - | Milli equivalents |
| N G T - | Nasogastric tube |
| NOR ml@ - ml/h - | Norepinephrine (in millilitres) at x millilitres per |
| | hour |
| NVALU BIOCHEM - | Normal values of biochemistry |
| NVALU PHYSICAL - | Normal values of clinical parameters |
| NVALU XR ECG - | Normal values related to x-ray and |
| | electrocardiogram |
| O2 - | Oxygen |
| PaCO2 - | Partial pressure of carbon dioxide in arterial blood |
| PaO2 - | Partial pressure of oxygen in arterial blood |
| PH - | Unit for measurement of acidity of alkalinity |
| PHENO - | Phenobarbitone |
| PHYS - | Physical |
| PEEP - | Peak end expiratory pressure |
| PIP - | Peak inspiratory pressure |
| PR - | Interval between P and R waves on ECG |
| PRE VER - | Prevertebral soft tissue shadow |
| QRS - | QRS complex on ECG |
| RES2 - | Resuscitation 2 |
| RESUS1 - | Resuscitation 1 |
| RESUS2 - | Resuscitation 2 |
| RE - | Rectal temperature |
| RR - | Respiratory rate |
| STYLET - | Stylet |
| SUCTION CAT - | Suction catheter |
| T⁰ OR - | Oral temperature |
| AX - | Axillary temperature |
| T—T - | Trachea to teeth distance |
| TV - | Tidal volume |
| U CAT - | Urinary catheter |
| UNCU - | Uncuffed |
| UREA/Cr - | Urea/creatinine |
| VENT - | Ventilation |
| XR ECG - | X-ray, electrocardiogram |
| VR - | Ventilation rate |

The manner in which a user navigates between the screens will now be described:

If the first screen is displayed and button 30 is pressed,then the second screen is displayed; if button 32 is pressed then the third screen is displayed; and if button 34 is pressed then the fourth screen is displayed.

If the second screen is displayed and button 30 is pressed, then the fifth screen is displayed; if button 32 is pressed then the sixth screen is displayed; and if button 34 is pressed then the first screen is displayed.

If the third screen is displayed and button 30 is pressed, then the seventh screen is displayed; if button 32 is pressed then the eighth screen is displayed; and if button 34 is pressed then the first screen is displayed.

If the fourth screen is displayed and button 30 is pressed, then the ninth screen is displayed; if button 32 is pressed then the tenth screen is displayed; and if button 34 is pressed then the first screen is displayed.

If the fifth screen is displayed and button 30 is pressed, then the second screen is displayed; if button 32 is pressed then the sixth screen is displayed; and if button 34 is pressed then the first screen is displayed.

If the sixth screen is displayed and button 30 is pressed, then the fifth screen is displayed; if button 32 is pressed then the second screen is displayed; and if button 34 is pressed then the first screen is displayed.

If the seventh screen is displayed and button 30 is pressed, then the third screen is displayed; if button 32 is pressed then the eighth screen is displayed; and if button 34 is pressed then the first screen is displayed.

If the eighth screen is displayed and button 30 is pressed, then the third screen is displayed; if button 32 is pressed then the seventh screen is displayed; and if button 34 is pressed then the first screen is displayed.

If the ninth screen is displayed and button 30 is pressed, then the fourth screen is displayed; if button 32 is pressed then the tenth screen is displayed; and if button 34 is pressed then the first screen is displayed.

If the tenth screen is displayed and button 30 is pressed, then the fourth screen is displayed; if button 32 is pressed then the ninth screen is displayed; and if button 34 is pressed then the first screen is displayed.

1. A paediatric electronic device which includes
a housing;
a length measuring means for measuring the length of a young child;
a memory means for storing data in regard to various weight dependent parameters;
a processing means for determining the value of at least one of the parameters in accordance with the length of the young child as measured by the length measuring means; and
a display means for displaying the values of the parameters.

2. The paediatric electronic device as claimed in claim 1, in which the parameters are grouped in various groups.

3. The paediatric electronic device as claimed in claim 2, which includes a group selecting means for selecting which group of parameters is displayed.

4. The paediatric electronic device as claimed in any one of the preceding claims, in which the parameters relate to physical measurements, suitable drugs, dosages, medical procedures, equipment, milestones or clothing.

5. The paediatric electronic device as claimed in any one of the preceding claims, in which the particular values of the various parameters for each length range are stored in a data base.

6. The paediatric electronic device as claimed in any one of claims 1 to 4, in which the particular values of the various parameters for each length are computed in accordance with a formula, stored in the memory means.

7. The paediatric electronic device as claimed in any one of the preceding claims, in which the memory means stores a length of a young child that has been measured, until reset.

8. The paediatric electronic device as claimed in any one of the preceding claims, in which the measuring means includes a length of tape housed in the housing and extractable therefrom, with an electronically operable monitoring means for monitoring the extent to which the tape has been extracted from the housing.

9. The paediatric electronic device as claimed in claim 8, which provides a retraction means to automatically retract the tape into the housing.

10. The paediatric electronic device as claimed in claim 8 or 9, in which a locking means is provided to lock the measuring tape when it has been extracted to a desired extent.
extracted from the housing after having been retracted into the housing.

10. The paediatric electronic device as claimed in any one of claims 6 to 9, characterised therein that the monitoring means includes an optical sensor (29) responsive to formations on the tape, the number of formations passing past the sensor being counted.

11. The paediatric electronic device as claimed in claim 10, in which the formations are openings (28) at spaced intervals in the tape.

12. The paediatric electronic device as claimed in any one of the preceding claims, in which the group selecting means includes a plurality of buttons (30, 32, 34) which are selectively depressed by the person using the device to select which group of parameters is to be displayed.

13. The paediatric electronic device as claimed in any one of the preceding claims, characterised therein that it includes a power supply (58) and a back-up power supply.

14. The paediatric electronic device as claimed in claim 13, characterised therein that it has a “sleep” function whereby the power consumption is reduced if the device is not operated for a specified time period.

15. The paediatric electronic device as claimed in claim 13 or 14, characterised

17. The paediatric electronic device as claimed in claim 15 or 16, in which a low voltage monitor is included together with a low voltage warning annunciator.

18. The paediatric electronic device as claimed in any one of the preceding claims, in which the display means includes a back-lit LCD display matrix with suitable drivers.

19. A paediatric electronic device substantially as herein described with reference to the accompanying drawings.
